(19) United States
(12) Patent Application Publication
(10) Pub. No.: US 2010/0288285 A1
(43) Pub. Date: Nov. 18, 2010

(54) TOOTHED VASECTOMY CLAMPS AND METHODS OF USING SAME

(76) Inventor: JOEL L. MARMAR, Philadelphia, PA (US)

Correspondence Address: ECKERT SEAMANS CHERIN & MELLOTT, 600 GRANT STREET, 44TH FLOOR, PITTSBURGH, PA, 15219, US

(21) Appl. No.: 12/834,551

(22) Filed: Jul. 12, 2010

Related U.S. Application Data
(63) Continuation-in-part of application No. 11/417,822, filed on May 4, 2006.

(51) Int. Cl. A61F 6/02

(52) U.S. Cl. 128/843

(57) ABSTRACT

Apparatus and methods for performing vasectomies are disclosed. In preferred embodiments, a clamp has a set of jaws, the distal ends of which terminate in a partial or completely circular structure that is essentially a split ring. The ring may include pointed tips structured to pierce the vas deferens. The tips engage opposing sides of the vas deferens. Various embodiments of the toothed clamps may be preferred for “open” versus “closed” vasectomies.

### CROSS REFERENCE TO RELATED APPLICATION

This application is a continuation-in-part and claims priority under 35 U.S.C. §119(e) to U.S. patent application Ser. No. 11/417,822, filed May 4, 2006, entitled, TOOTHED VASECTOMY CLAMPS AND METHODS OF USING SAME.

### BACKGROUND OF THE INVENTION

1. Field of the Invention

The present invention relates to surgical instruments, and more particularly to toothed clamps for use in vasectomy procedures.

2. Background of the Invention

Male sterilization via surgery is often accomplished via a vasectomy, namely that involves removal or disruption of at least a portion of the vas deferens (hereinafter, the “vas”). Currently, there 2 ways to access the vas prior to the occlusion process. “Open” vasectomies are performed by making standard scrotal incisions first, then securing the vas through the opening created by the incisions. “Closed” procedures, or “no-scalpel” vasectomy procedures, however, have also been developed, whereby, the scrotal skin with the vas below are secured together with a round clamp before the skin is opened with a sharp pointed hemostat. The same dissecting hemostat is used again to “skewer” the vas and elevate it above the skin surface. No scalpel procedures are an improvement since incisions/openings into the scrotum are minimized and the attendant apprehension of the patient is reduced. The no-scalpel technique is less invasive and may be accomplished in a shorter time than a traditional vasectomy, but also presents challenges for the surgeon. For example, the round clamp may slip in men with thick scrotal skin and there is a long learning curve for the use of the pointed hemostat.

For example, in each case, the no-scalpel vasectomy (closed access) and standard vasectomies (open access) require that the vas be palpated and identified before any skin opening is made. Then, in the case of “closed” access, the vas and surrounding skin of the scrotum is fixed securely in position with a ringed clamp, or else with “open access, the vas and surrounding fascia are grasped after the skin opening is completed. As described in Marmar et al., “A Minimally Invasive Vasectomy With the No Suture, Inline Method for Vas Occlusion,” (Int. J. Fertil. 46(5):257-264, 2001) this is another “open” access surgical alternative. Although the treatment of the vas is quick and effective with this method, the vas and fascia must still be grasped securely to perform the “Inline Vasectomy.”

Regardless of the vasectomy method, a hemostat or knife pierces the scrotum at some point, but the vas with overlying scrotal skin is grasped and secured with an encircling clamp as part of a “closed” access procedure and the vas is elevated above the skin level thru the skin opening with the dissecting hemostat or other instrument. For “open” access, the skin is opened first, and then the vas is secured by a clamp. It is important to note that in the initial reporting of the In Line Vasectomy by Marmar et al, 2001, a segment of the vas is selectively secured by a round clamp, and then raised by a skin hook, cut and then ligated/clipped and cauterized. Each step of the procedure requires different surgical tools to accommodate a relatively high degree of surgical skill and dexterity needed for this procedure. The toothed vasectomy clamps reported in this patent application represent greater reliability over other clamps for securing the vas in both “closed” and “open” procedures, and these clamps will lead to the need for fewer instruments, overall.

The vas is an elusive structure and it must be held in place throughout the procedure since it will tend to immediately recoil into the scrotum whenever it is not secured by a clamp. This is made more difficult due to the nature of the vas as being somewhat rigid, i.e. the vas does not collapse/deform in the manner of a softer vessel. As such, devices such as, but not limited to, hemostats and forceps are not well structured to grasp and hold a vas. That is, in relation to holding a vas, surgical tools structured to merely compress are not as effective as tools structured to pierce. Other instruments with teeth are available, but they present some problems. For example, round clamps with or without teeth are not used for “open” vasectomies because they may injure the posterior mesentery with the blood supply to the vas when the clamp encircles the entire vas. Forceps with teeth may secure a portion of the vas during an “open” vasectomy, but they require continuous pressure of the thumb and forefinger, and this action may become tiresome to the surgeon, and as a result, the vas may slip away. The loss of the vas after the start of the procedure will require extensive dissection and manipulation often resulting in increased bleeding and swelling. Thus, any tool or device that simplifies these procedures by providing firm fixation of the vas would be desirable.

Tools specifically for use in vasectomy procedures are well known, for example, U.S. Pat. No. 5,067,958, Sandhaus, discloses a device for use in a vasectomy that has non-circular, asymmetrical jaws. U.S. Pat. No. 4,920,982, but this device is primarily intended to deliver a clip to the vas deferens. Goldstein discloses a clamp having a circular jaw (FIG. 1) with an opening on the proximal side of the clamp end that is provided. This gap is provided so that no undue force is exerted on the scrotal skin. (Col. 3, lines 49-59). Therefore, although this device is used in a vasectomy procedure, it is used during a different type of procedure and for a different purpose than the invention. That is, the Goldstein clamp is used during a “closed” vasectomy procedure, wherein the vas is not lifted above the skin by the clamp, as opposed to an “open” vasectomy procedure, wherein the scrotal skin is cut and the then vas is secured and lifted thru the opening and above the skin by the same instrument. Furthermore, even for “closed” vasectomies, the Goldstein clamp does not have teeth and has been known to slip in some cases. It is noted that “open” and “closed” procedures, although broadly similar, are different procedures and each requires different surgical tools.

Additionally, it is noted that the vas has a corresponding artery, the vassal artery, as well as other blood vessels that extends along the vas within the scrotum. The vasal artery is, typically, disposed on the posterior (inner) side of the vas and it is contained within a mesentary. This artery supplies the vas, as well as the testes and other downstream organs, etc. with blood. That is, the plurality of smaller blood vessels extend upward within the mesentery to supply the vassal artery and the vas. If the vasal artery is compressed or disrupted, blood flow therethrough is reduced which may cause injury, typically bleeding or hematoma formation during the procedure. It is further noted that surgical tools that completely encircle the vas during “open” access procedures may be undesirable because of they may disrupt or damage the posterior mesentery and the vasculature behind the vas. Accordingly, it is desirable to not clamp or pierce the vasal artery, or any of the smaller blood vessels, during the procedure. Hereinafter, the phrase “vasal artery” shall refer to both the vasal artery as well as any blood vessels that couple the vasal artery to the vas.

None of these prior art devices, i.e. round clamps that completely encircle the vas without teeth, however, permits these procedures to be performed in an effective and efficient manner and with the confidence that the vas will not slip during the procedure and that the clamp will not disrupt the vasal artery. Therefore, there remains a long-felt yet unmet need for providing devices specifically designed to facilitate a “no scalpel,” “in line” and other vasectomies. It would further be desirable to provide such improvements in a manner that permitted their application across a variety of situations and that permitted their implementation in a cost-effective manner.

### SUMMARY OF THE INVENTION

Accordingly, it has now been found that these and other shortcomings of the prior art can be overcome by providing a clamp which has a set of jaws and teeth, the distal end of which terminate in a clamp assembly which is a circular structure that is essentially a split ring. As part of the round clamps, each jaw has a serrated edge or a pointed tip, and each jaw is slightly less than a half-circle so that when closed upon the vas there is a small slit opening toward the proximal side of the jaws. These round clamps with teeth are preferred for “closed” access procedures, because they will not slip. The circular jaws are adapted to grasp the vas and the overlying skin of a patient and the slit permits a scalpel blade, scissors or cautery to cut an opening in the scrotal skin. In contrast, the mini tenaculum is preferred for “open” procedures. After this mini tenaculum is applied to the upper ½ of the vas it permits the surgeon to cut the vas sheath longitudinally in the clamped region. Moreover, the clamp assembly is structured to engage opposing sides of the vas. In this configuration, there is a reduced chance of the vassal artery being compressed or pierced. That is, the clamp assembly is, preferably, structured/sized so that it cannot completely encircle the vas, but at the same time, after the vas is secured, it may be raised above the skin level by a downward rotation of the handle. For example, in a preferred embodiment, the clamp assembly has two opposing clamp components wherein each component has an arcuate member medial portion that is a quarter of a circle. Distal tips extend inwardly at the distal end of the medial portion. The arcuate portions, i.e. the quarter circular arcs, are sized to fit partially around a vas. That is, the quarter circular arcs are the size of a typical vas and, as such, the clamp assembly does not encircle the entire vas, but rather the distal ends engage and pierce the opposing sided of, about, the upper ½ of the vas. In this configuration, the clamp assembly engages the vas at a location spaced from the vasal artery and mesentery. When the vas is secured by this clamp, the vas can be raised above the skin surface by a downward rotation of the handle.

It is known to provide surgical tools which have first and second members connected at a pivot point that is movable between a closed position and an open position. Each of these members has distal and proximal ends. Each of the proximal ends preferably has a handle. Each of the distal ends, preferably, has a component that is an at least partially arcuate portion that has a less than semi-annular section and terminates at a toothed tip. The two components form a clamping structure. The components each have a proximal portion and a distal portion.

In accordance with certain aspects of the present invention, these clamping structure component distal portions are structured to engage opposing side of the vas. The clamping structure component distal portions may be tapered and/or sharp, i.e. pointed, and further structured to pierce the vas. When the clamping structure component distal portions are pointed, the clamping structure component distal portions are structured to pierce as well as partially encircle the upper ½ of the vas. This configuration provides for a secure grip on the vas. The proximal portions of the semi-annular sections, however, remain spaced apart from one another to form a slot so that a clamping structure is formed that less than completely encircles a body structure, which in a most preferred embodiment is a vas. In preferred embodiments, the slot remaining between the clamping structure component proximal portions is between 1.0 and 2.0 mm wide.

In certain other embodiments the clamping structure component distal portions have opposing surfaces with a plurality of teeth that are engageable with one another to form a closed joint. These clamps are preferred for “closed” vasectomies.

In the preferred embodiment, however, the surgical apparatus of the present invention comprises first and second members connected at a pivot point that is movable between a closed position and an open position. Each of these members has distal and proximal ends, and each of the proximal ends preferably has a handle. Each of the distal ends, i.e. the clamping structure components, preferably has an at least partially arcuate portion that has a less than semi-annular section and terminates at a pointed tip, such that the distal end of the first member and the distal end of the second member remain spaced apart from one another when the apparatus is in the closed position and the semi-annular sections remain spaced apart from one another in the closed position to form a slot, whereby a clamping structure is formed that less than completely encircles a body structure. In such embodiments, it is preferred that the first and second members includes a pointed hook portion as part of the distal ends and the hook portions of the distal ends can be either angled or straight. However, it is again preferred that the proximal portion of the clamping structure components are spaced apart between 1.0 and 2.0 mm when the apparatus is in the closed position.

The present invention also relates to improved methods for performing a vasectomy that use a vas clamp comprising: first and second members pivotably connected to one another and movable between a closed position and an open position, each of the first and second members having oppositely disposed proximal and distal ends, wherein each of the distal ends includes an arcuate portion. The surgeon then moves the first and second members to the closed position around a vas, thereby grasping opposing sides of the vas so that in the closed position the first member and the second member do not completely encircle the vas and leave a slot on a side of the distal ends toward the proximal end of the first and second members. The vas is then pulled above skin level thru the opening in the scrotal sac and cut, completing the vasectomy. In such methods, the distal ends either may also abut, thereby leaving a single slot on the side of the distal tips toward the proximal end of the clamping structure components, or remain spaced apart.

It is further noted that during a “closed” vasectomy procedure, as opposed to a “open” vasectomy procedure, the vas is more difficult to grip due to the thick scrotal skin. That is, in very general terms, the scrotal skin and the vas below may be too thick to grasp and secure by a round clamp without teeth. In contrast, the use of a round clamp with teeth far is more reliable for gripping and securing the scrotal skin with the vas below. As such, when performing an “open” vasectomy procedure it is desirable for the surgical apparatus to hook, that is, pierce the skin, and the vas below so as to substantially reduce the chance of the vas slipping through the surgical apparatus. Further, given that the vasal artery is disposed behind the vas, it is preferable that the vas clamp is structured to engage opposing sides of the upper ½ of the vas. That is, to avoid the clamp disrupting the vasal artery, the vas clamp is used to grip/pierce opposing lateral sides of the scrotal skin over the vas.

Further, as the vas clamp preferably does not close at the proximal side of the distal ends, the vas clamp may include a stop structured to prevent full closure of the distal end. That is, and as is known, the vas clamp first and second members may have an abutting surface at the pivot point or a locking structure adjacent the handles which is structured to prevent the vas clamp distal ends from closing or crossing the midline.

### BRIEF DESCRIPTION OF THE DRAWINGS

FIG. 1 is a plan view of a multi-tooth vas clamp made in accordance with the present invention;

FIG. 2 is a side elevation view of the vas clamp shown in FIG. 1;

FIG. 3 is an enlarged elevation view of the distal end of the vas clamp shown in FIGS. 1-2;

FIG. 4 is a plan view of the distal end of the vas clamp shown in FIG. 3;

FIG. 5 is a front view of the distal end of the vas clamp shown in FIG. 4;

FIG. 6 is a plan view of a single tooth vas clamp made in accordance with the present invention;

FIG. 7 is a side elevation view of the vas clamp shown in FIG. 6;

FIG. 8 is an enlarged elevation view of the distal end of the vas clamp shown in FIGS. 6-7;

FIG. 9 is a plan view of the distal end of the vas clamp shown in FIG. 8;

FIG. 10 is a front view of the distal end of the vas clamp shown in FIG. 9;

FIG. 11 is a diagrammatic view of a vasectomy procedure being conducted using the vas clamp of the present invention;

FIG. 12 is a plan view of a single tooth tenaculum vas clamp made in accordance with the present invention;

FIG. 13 is an enlarged plan view of the distal end of the vas clamp shown in FIG. 12;

FIG. 14 is a plan view of an angled single tooth tenaculum vas clamp made in accordance with the present invention;

FIG. 15 is an enlarged plan view of the distal end of the vas clamp shown in FIG. 14;

FIG. 16 is a plan view of a half-tenaculum vas clamp made in accordance with the present invention;

FIG. 17 is an enlarged plan view of the distal ends of the vas clamp shown in FIG. 16;

FIG. 18 is a plan view of the vas clamp shown in FIG. 16 in the closed position;

FIG. 19 is a plan view of an angled multi-tooth half-tenaculum vas clamp made in accordance with the present invention;

FIG. 20 is an enlarged plan view of the distal ends of the vas clamp shown in FIG. 19;

FIG. 21 is an enlarged elevation view of a first distal end of the vas clamp shown in FIG. 19;

FIG. 22 is an enlarged elevation view of a second distal end of the vas clamp shown in FIG. 19;

FIG. 23 is a plan view of the vas clamp shown in FIG. 19 in the closed position;

FIG. 24 is an isometric view of the vas clamp of FIG. 12 in use; (this figure demonstrates how the clamp pierces the side walls of the vas securely, but does not go behind the vas to disrupt the posterior mesentery with the blood supply to the vas)

FIG. 25 is an enlarged view of the tip of the vas clamp shown in FIG. 24; and

FIG. 26 is a flow chart of the associated method.

### DETAILED DESCRIPTION OF THE PREFERRED EMBODIMENTS

The implementation of the present invention is in several preferred embodiments, discussed below, along with several illustrative examples. The embodiments of the invention described below are provided for the purpose of understanding the invention and are not meant to be limiting.

The clamp 100, described below, is structured to be used by a human hand. “Distal” and “proximal” are relative terms. Accordingly, as used herein, “distal” means located away from the user's hand and “proximal” means located closer to the user's hand.

As used herein, “structured to allow a scalpel to pass therethrough,” in reference to a gap means that the surgeon has enough room to manipulate the scalpel as desired, not merely enough room to pass the scalpel through the gap. Preferably, such a gap is at least 1.0 mm wide.

As used herein, an element “partially encircling” an object means that the element abuts a portion of the perimeter of the object but does not enclose the object. That is, at the location of the element, a portion of the object is exposed. Such a portion is larger than a gap “structured to allow a scalpel to pass therethrough” noted above. For example, and as described below, a clamp that partially encircles a tubular object, such as the vas, extends over a portion, e.g. about half or 75% of the perimeter of the vas, while leaving a portion of the vas exposed.

As used herein, a “semicircular” portion extends over an arc of about 180 degrees.

As used herein, a “quarter-circular” portion extends over an arc of about 90 degrees.

As used herein, any radius measurement refers to the inner diameter, i.e. inner surface, of any arcuate member.

As used herein, the “centerline” of an arcuate tip is a line extending generally tangent to the arc at the tip.

As used herein, “opposing sides” of a generally tubular element are the portions of a circumference on opposite sides of a line representing a diameter.

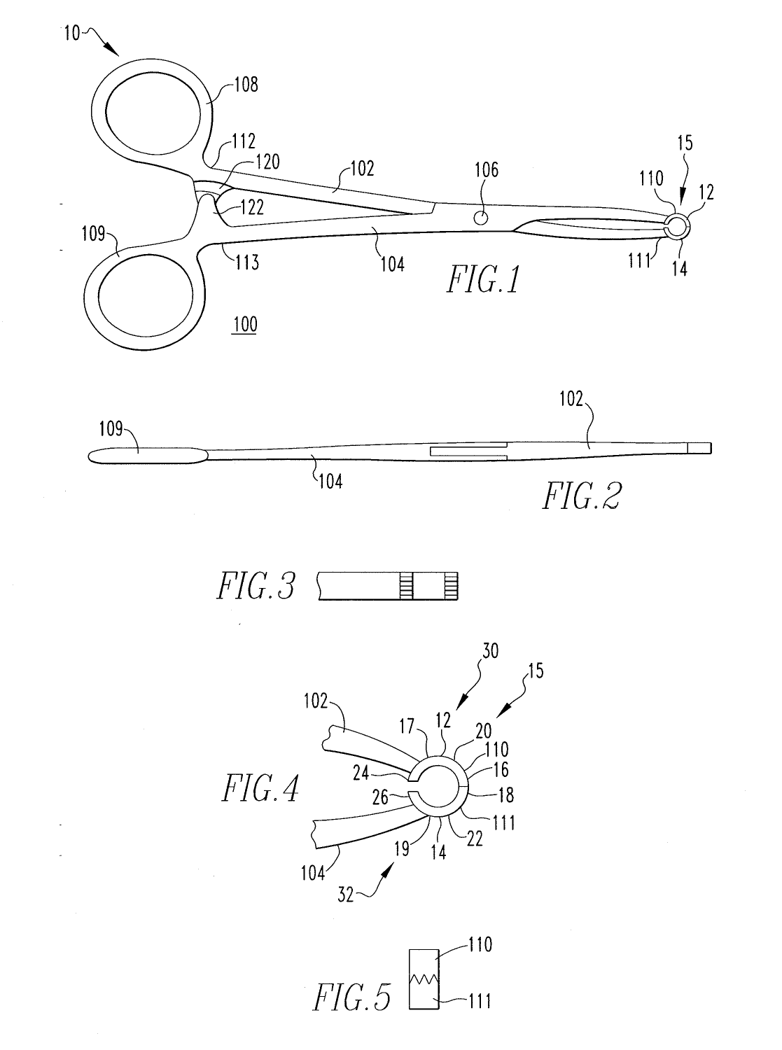

The general design and construction of the surgical apparatus 10, which is preferably a vas clamp 100, 200, 300, 400, 500, 600 (each discussed below) disclosed herein will be familiar to those skilled in the art. The selection of materials and overall size and shape of these surgical instruments is similarly well known. Referring now to FIG. 1, a plan view of a multi-tooth vas clamp 100 made in accordance with the present invention is shown. As is typical, the clamp is comprised of a first member 102 and a second member 104 that are connected by a pivot point 106. The pivotable connection permits the clamp 100 to be opened and closed, as described in further detail below. The first member 102 and second member 104 each have a proximal end 112, 113 (respectively) that terminate in a handle portion 108, 109 that is illustrated as the traditional finger loop handle, however, this structure is not part of the present invention. Similarly, the first member 102 and a second member 104 each comprise, respectively, a locking portion 120, 122 that permits the clamp to be locked in a closed position, most preferably under two or more degrees of varying pressure depending upon which of two or more locking positions is chosen by the surgeon, as known in the art. As seen in the elevation view of FIG. 2, the vas clamp of FIG. 1 is substantially straight and has only a minor taper toward its distal end. In one preferred embodiment, the overall length of the vas clamp 100 illustrated in FIGS. 1-2 is about 10.5 cm.

Referring again to FIG. 1, it can be seen that the first member 102 and second member 104 terminate at their respective distal ends 110, 111. In accordance with the present invention, these distal ends 110, 111 each comprise a portion of a clamping structure 15 is most preferably about one-half the clamping structure 15, although it is not necessary that the distal ends 110, 111 be symmetrical or of equal size, as will be appreciated with reference to alternate preferred embodiments described in detail below. The clamping structure 15 includes a first component 17 and a second component 19. The clamping structure first component 17 is disposed at the first member distal end 110 and the clamping structure second component 19 is disposed on the second member distal end 111. The clamping structure 15, that is the two components 17, 19, are structured to engage opposing sides of a tubular body member, such as, but not limited to a vas 50.

In the embodiment illustrated in FIG. 1, the clamping structure 15 is substantially ring-like except for an opening on the rear, or toward the distal side of the clamping structure 15. This clamp is preferred for “closed access procedures that engage the overlying skin and the vas below, together. The teeth, discussed below, of this clamp secure the structures without slippage. In one preferred embodiment, the clamping structure 15 has an outside diameter of approximately 0.5 cm and an inside diameter of approximately 0.4 cm. As explained in further detail below, the provision of this opening permits improvements in the vasectomy procedure; that is, the gap on the proximal side of the clamping structure 15 is structured to allow a scalpel to pass therethrough. The gap is between about 0.5 and 3.0 mm, and more preferably about 1.0 mm.

Further details of the vas clamp 100 illustrated in FIG. 1 are visible in FIGS. 3-5. FIG. 3 is an enlarged side elevation view of the distal ends 110, 111 of the vas clamp 100 shown in FIG. 1 and illustrates the serrations or teeth provided in this particular embodiment. In one preferred embodiment, the thickness of the distal ends 110, 111 is between about 1.75-2.0 mm and comprises three teeth. The engagement of these teeth is illustrated in FIG. 5. Referring now to FIG. 4, a plan view of the distal end 110, 111 of the vas clamp 100 shown in FIG. 3 is shown along with a broken away portion of the first member 102 and second member 104. As seen in FIG. 4, the opening between the distal ends 110, 111 discussed above with reference to FIG. 1 is clearly seen. In one preferred embodiment, this opening is about 1.0 mm across.

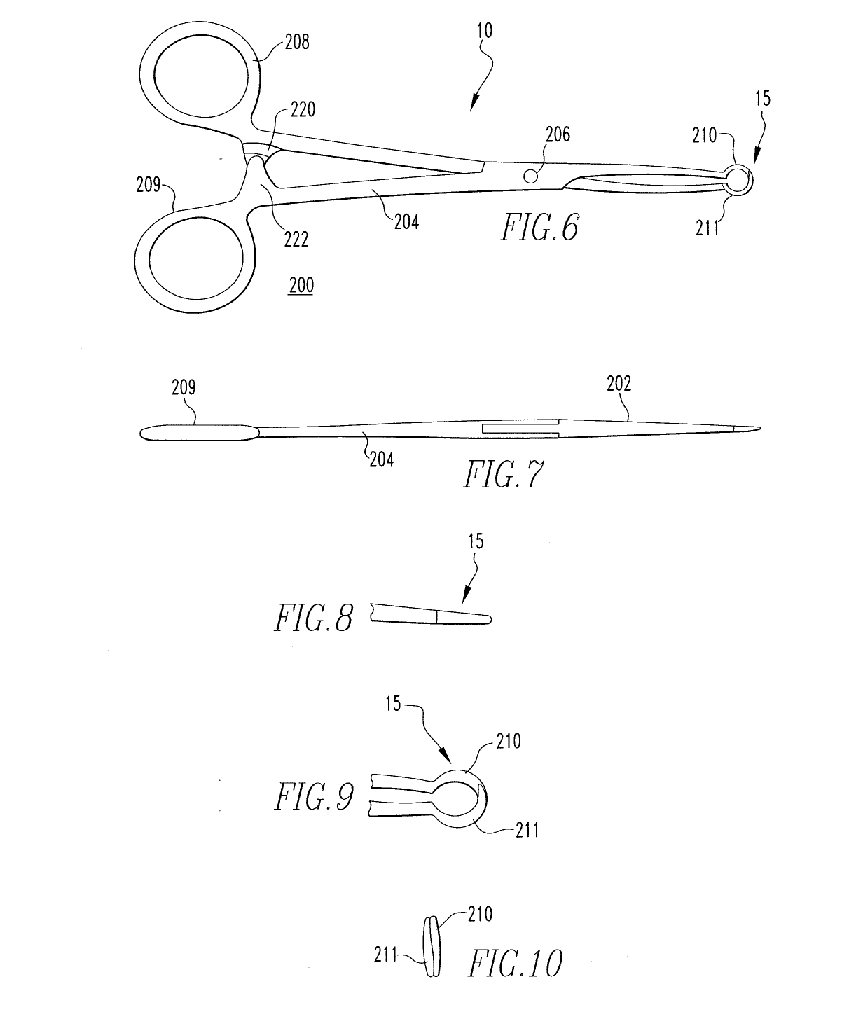

Another preferred embodiment of the present invention is illustrated in FIG. 6, which is also preferred for “closed” access procedures This embodiment the distal end of the clamp 200 has a single tooth. As described above, the embodiment of FIG. 6 also comprises first and second members 202, 204 connected by a pivot 206 and terminating at their respective proximal ends with handle portions 208, 209 and including locking portions 120, 122. As seen in FIG. 7, unlike the embodiment described above, the first and second members 202, 204 taper as they approach the distal ends 210, 211.

Further details of the single tooth vas clamp illustrated in FIG. 6 are visible in FIGS. 8-10. FIG. 8 is an enlarged side elevation view of the distal ends 210, 211 of the vas clamp 200 shown in FIG. 6 and illustrates the single tooth provided in this particular embodiment. In one preferred embodiment the thickness of the distal ends 210, 211 is about 1.0 mm. The engagement of these teeth is illustrated in FIG. 10, as illustrated; the curved, arcuate teeth are tapered and overlap. In one preferred embodiment, the overall thickness of this overlapped structure is about 1.5 mm. Referring now to FIG. 9, a plan view of the distal ends 210, 211 shown in FIG. 8 is shown along with a broken away portion of the first member 202 and second member 204. As seen in FIG. 9, the opening between the distal ends 110, 111 discussed above with reference to FIG. 1 is clearly seen. In one preferred embodiment, this opening is about 1 mm (0.1 cm) across.

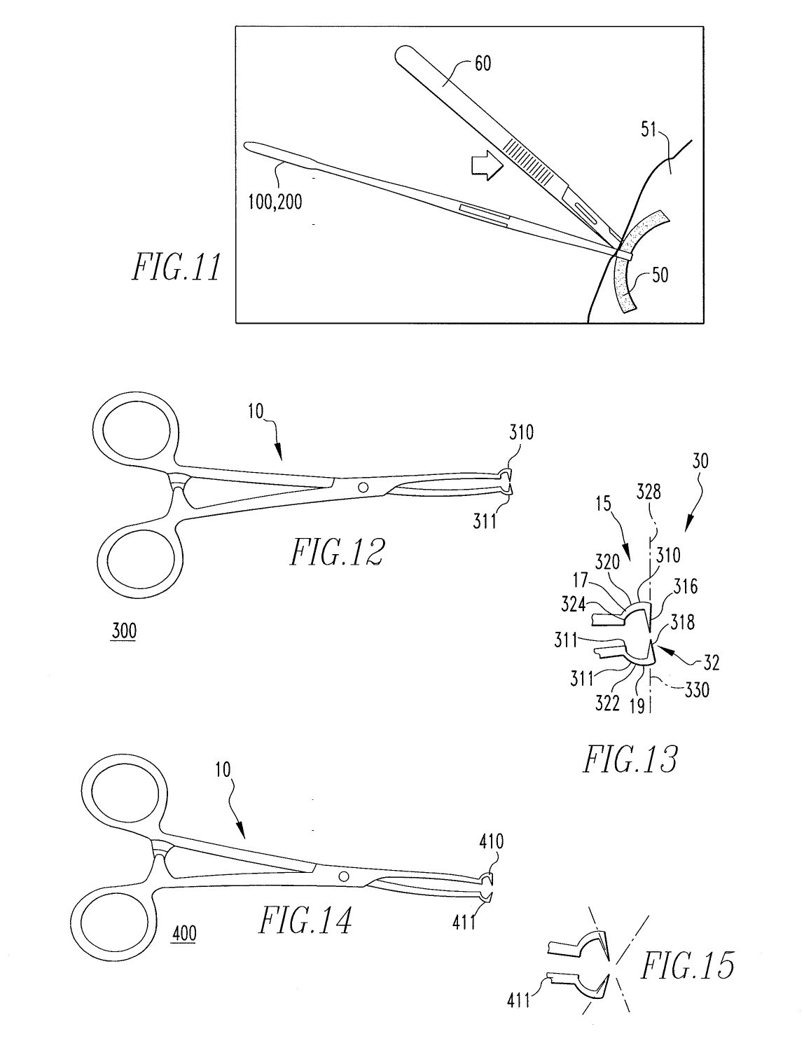

FIG. 11 is a diagrammatic view of a “closed” vasectomy procedure being conducted using the vas clamp 100 of the present invention. As explained above, the use of such clamp is not an important part of “in line” or standard “open” vasectomy. The toothed vas clamp 100, as well as other embodiments disclosed herein, have the ability to firmly grasp the vas and the overlying skin, and to continually manipulate this duct at the start of the “closed” procedure, thereby minimizing the risk of slippage, the extent of the dissection and reducing the time of the procedure. In FIG. 11, the procedure is to a point where the vas 50 has been grasped by a vas clamp 100, 200 made in accordance with the present invention. The curved, arcuate distal ends enable a firm grasp without crushing the vas 50 and overlying skin 51 while the opening described above on the proximal side is large enough to permit incising the vas using a scalpel 60, most preferably carrying a #15 blade. As a result of both the firm grasp and the ability to make a clean longitudinal incision provided by the slotted opening, the vasectomy procedure can be readily completed with less time, trauma and chance of error.

Additional embodiments of the present invention are disclosed in FIGS. 12-15. These clamps are preferred for “open” vasectomies. The teeth at the distal end will pierce and secure the upper ½ of the vas without disruption to the posterior mesentery with its blood supply. Referring now to FIG. 12, a plan view of a single tooth tenaculum vas clamp 300 made in accordance with the present invention is shown. This clamp 300 is made substantially in accordance with the structures described above with reference to FIGS. 1-10; however, as see in FIG. 12, the distal ends 310, 311 may or may not touch to form a closed grasping structure, as described above. Moreover, the distal ends 310, 311 of this embodiment are pointed ends hooked so they perform a hooking function during the “open” vasectomy procedure. Further details of the distal ends 310, 311 are shown in FIG. 13, which is an enlarged plan view of the distal end of the vas clamp 300 shown in FIG. 12. In a preferred embodiment, the respective arcuate sections have a radius of about 2.5 mm and from the tip of the hook to the bottom of the curve is about 1.5 mm on each side, thus leaving a 2.0 mm gap between the tips of the hook.

A variation of this device for “open” vasectomy procedures is shown in FIG. 14 is a plan view of an angled single tooth tenaculum vas clamp 400 made in accordance with the present invention. Unlike the vas clamp 300 shown in FIGS. 12-13, this embodiment has tips that are angled, in other words, the axes of the most distal potion of the distal ends 410, 411 intersect, as shown by the dashed lines in the enlarged plan view of the distal end of the vas clamp 400 shown in FIG. 15. The tips of this clamp will secure the lateral walls of the vas in those “open” vasectomy cases with a thick fascia overlying the pure vas. The slightly angled tips can engage the side walls of the vas and accommodate for the extra girth of the thick fascia.

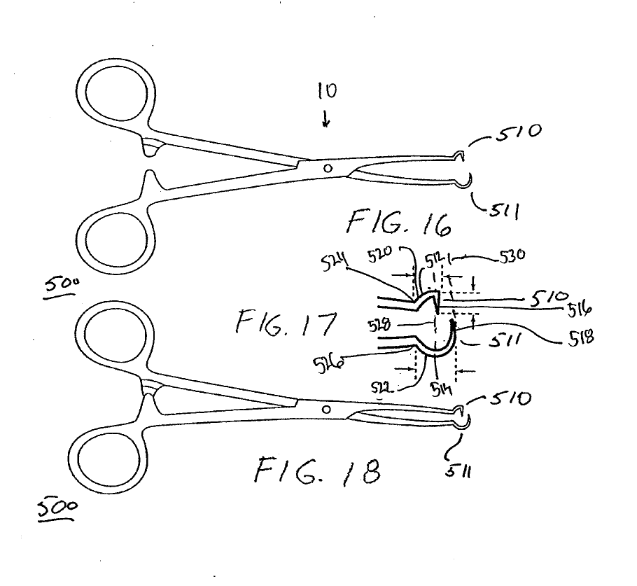

A different embodiment of the present invention is disclosed in FIGS. 16-18. Referring now to FIG. 16, a plan view of a half-tenaculum vas clamp 500 made in accordance with the present invention is shown, but it is preferred for a “closed” vasectomy in a man with very thick scrotal skin. This clamp 500 is made substantially in accordance with the structures described above with reference to FIGS. 1-15. However, as see in FIG. 16, the distal ends 510, 511 do not touch to form a closed grasping structure, as described above; however, unlike the other preferred embodiments, one distal end 510 has a different shape than the other distal end 511. With this shape, the clamp will provide more room to accommodate a patient with very thick skin for a “closed” vasectomy. As described immediately above, the distal ends 510, 511 of this embodiment have pointed ends that are shaped so they perform a hooking function during the procedure; however, they are not symmetrical. Further details of the distal ends 510, 511 are shown in FIG. 17, which is an enlarged plan view of the distal end of the vas clamp 500 shown in FIG. 16. In a preferred embodiment, one of the respective arcuate member medial portion 520, a quarter-circular portion, has a radius of about 2.5 mm and from the tip of the hook to the bottom of the curve is about 1.5 mm. This end 510 is structured to pierce both the scrotum and possibly the vas 50. The other distal end 511 is a fuller “hook,” i.e. a semicircular portion, preferably has a radius of about 2.5 mm, but because the arcuate portion is more complete, i.e. the arcuate portion is longer, the hook has a width of about 5 mm. As seen in FIG. 18, when this pair of distal ends 510, 511 is closed, the pointed tip of the first distal end 510 is located at about the center of the arc described by the second distal end 511. This pointed tip will pierce the skin and lateral wall of the vas securely, while the fuller “hook” will engage the very thick skin. When the clamp is rolled on its side the overlying skin will be in a secure position to begin a “closed” vasectomy despite its thickness.

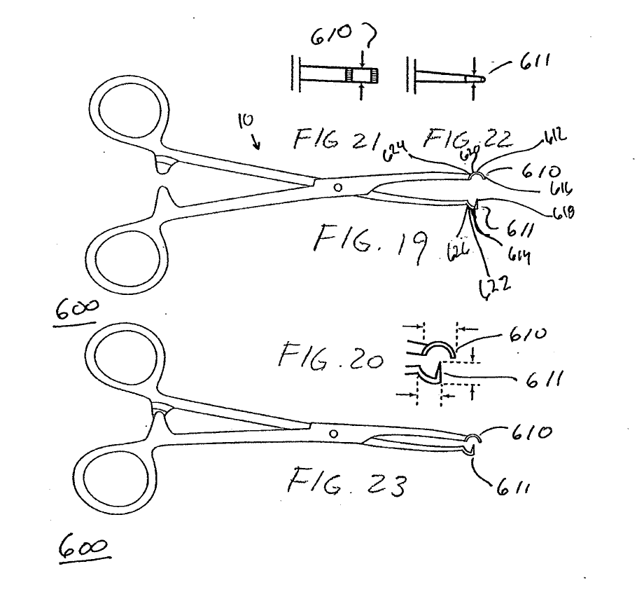

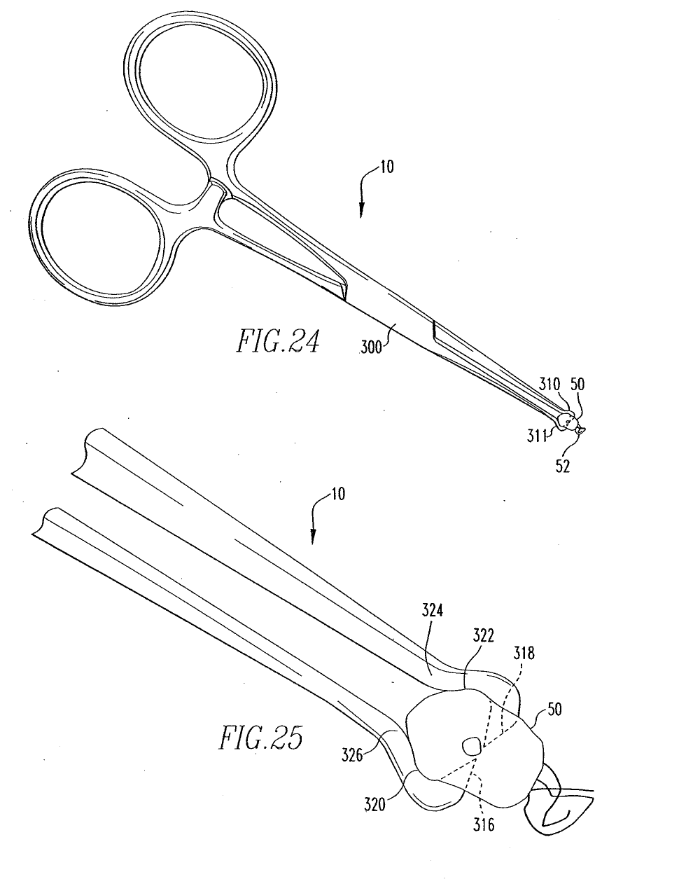

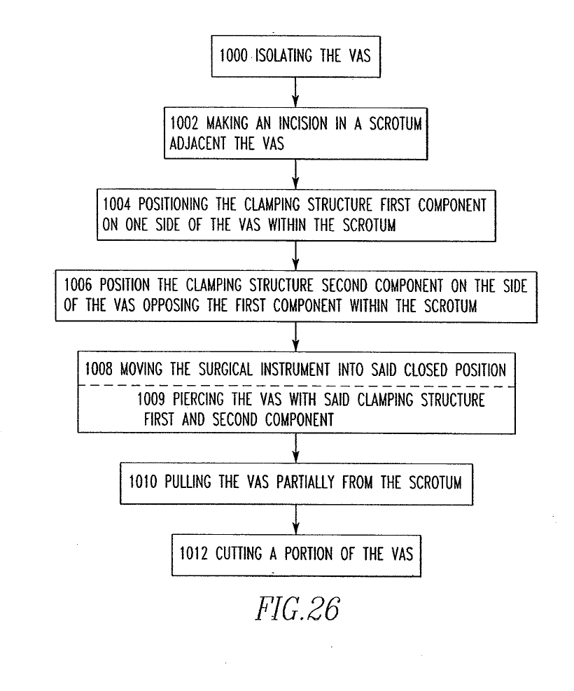

A further alternate embodiment of the present invention is disclosed in FIGS. 19-23. Referring now to FIG. 19, a plan view of a multi-tooth half-tenaculum vas clamp 600 made in accordance with the present invention is shown. This clamp 600 is made substantially in accordance with the structures described above with reference to FIGS. 1-18, but it is preferred for “closed” vasectomies on men with very thick skin. As seen in FIG. 19 and in the enlarged plan view of the distal ends 610, 611 of the vas clamp 600 shown in FIG. 20, as described above with reference to FIGS. 16-18, the distal ends 610, 611 are not symmetric. A first distal end 610 is an arcuate section with a width of about 5 mm, while the second distal end 611 is a half tenaculum, as described above. Thus, as illustrated in FIG. 23, as with the embodiment described with reference to FIG. 18, the distal ends 610, 611 of the jaws align in the closed position so that the point of the tenaculum that terminates the second distal end 611 is approximately centered over the arcuate distal end 610. As best illustrated in the elevation views of FIGS. 21-22, the distal ends 610, 611 further differ from the embodiments discussed immediately above in that the arcuate first distal end 610 has a multi-tooth jaw.

The embodiments set forth above each have similar, clamp structures 15 and will be discussed in more detail below. As shown in FIG. 4, the vas clamp distal ends 110, 111, that is the clamping structure 15, include opposing arcuate members 12, 14. The arcuate members 12, 14 each have a distal tip 16, 18 (respectively), a medial portion 20, 22, and a proximal tip 24, 26. The first and second members 102, 104 are coupled to the arcuate member medial portions 20, 22. More specifically, as shown in FIG. 1, the first and second members 102, 104 have a handle portion 108, 109 (as noted above) as well as two jaw portions 30, 32 disposed on the other side of the pivot point 106, i.e. between the pivot point and the distal ends 110, 112. As is known, the jaw portions 30, 32 move in a scissor-like motion corresponding to a user actuation on the handle portions 108, 109. The jaw portions 30, 32 are, preferably, elongated and may be generally semicircular. That is, the medial section of each jaw portion 30, 32 arc away from each other before arcing toward each other at the distal ends 111, 110.

In this configuration, the clamp structure 15 is, and more specifically the distal tips 16, 18 are, structured to engage the scrotal skin while surrounding the vas and maintaining a scalpel accessible gap between the distal and proximal tips 24, 26. Thus, if positioned to abut each other, the clamp mechanism 15 would form a substantially complete, but partial torus. More specifically, as noted above, there is a gap between the arcuate member proximal tips 24, 26 structured to allow a scalpel 60 to pass therethrough.

The embodiment shown in FIGS. 12 and 13 has been effective in providing a secure grip of a vas 50, and it is specifically preferred for “open” vasectomies. Unlike the embodiment described above, the clamping structure 15 includes the shaped distal ends 310, 311 shown in FIG. 13 which do not form a substantially complete but partial torus, but rather a half torus, i.e. the first and second components 17, 19 are each about a quarter-circle and together form, generally, a semicircle. That is, in this embodiment, clamping structure first and second components 17, 19 are the distal ends 310, 311 which include a quarter-circular medial portion 320, 322, an inwardly extending (that is, extending toward the center defined by the arcuate medial portion 320, 322) distal tip 316, 318, and a proximal end 324, 326. In the closed position, this clamp will pierce and secure only the upper ½ of the vas and not disrupt the posterior mesentery with the blood supply to the vas.

The inwardly extending distal tips 316, 318 are generally straight and pointed, and each has a centerline 328, 330. The inwardly extending distal tips 316, 318 may be aligned with each other, that is, the distal tips 316, 318 share a substantially common centerline 328, 330 (FIG. 12). The inwardly extending distal tips 316, 318 may be offset but parallel to each other, that is, the distal tips 316, 318 do not share a common centerline, but the two centerlines 328, 330 are parallel (FIG. 17). The inwardly extending distal tips 316, 318 may be angled relative to each other (FIG. 15). In all embodiments, the two centerlines 328, 330 are, preferably, substantially in the same plane. The pointed distal tips 316, 318 are structured to pierce a vas. Moreover, the medial portion 320, 322 of the distal ends 310, 311 of this embodiment are generally arcuate and are sized to partially encircle a vas. That is, the arcuate medial portions 320, 322 have a radius of between about 1.0 and 3.0 mm, and more preferably about 2.0 mm. As before, the proximal ends 324, 326 have a gap therebetween, even when the clamp 300 is in the closed position.

In the embodiment shown in FIGS. 16 and 17, the clamping structure 15, includes opposing arcuate members 512, 514. The arcuate members 512, 514 each have a distal tip 516, 518 (respectively), a medial portion 520, 522, and a proximal tip 524, 526. As before, first and second members 512, 514 are coupled to the arcuate member medial portions 520, 522. The clamping structure first and second components 17, 19 are the distal ends 510, 511. In this embodiment, the clamping structure first component, 17 arcuate member 512, has a quarter-circular medial portion 520 with an inwardly extending, pointed distal tip 516. The clamping structure second component 19 has a semicircular medial portion 522 with an arcuate pointed distal tip 518. In this embodiment, the first distal tip centerline 528 and said second component distal tip centerline 530 are parallel and offset from each other.

In the embodiment shown in FIGS. 19 and 20, the clamping structure 15, includes opposing arcuate members 612, 614. This clamp is preferred for “closed” vasectomy procedures on men with very thick scrotal skin. The arcuate members 612, 614 each have a distal tip 616, 618 (respectively), a medial portion 620, 622, and a proximal tip 624, 626. As before, first and second members 612, 614 are coupled to the arcuate member medial portions 620, 622. The clamping structure first and second components 17, 19 are the distal ends 610, 611. In this embodiment, the clamping structure first component, 17 arcuate member 612 has a quarter-circular medial portion 620 with an inwardly extending, pointed distal tip 616. The clamping structure second component 19 has a semicircular medial portion 622 with a flat distal tip 618.

In use, this embodiment allows the surgeon at the start of a “closed” vasectomy to securely grasp the overlying, very thick scrotal skin and the vas 50 below. That is, the surgeon positions the distal tips 516, 518 on opposing sides of the vas 50, at a location spaced from the vasal artery 52. Upon actuation of the handles, the distal tips 516 pierces the vas 50 from one lateral sides, i.e. spaced from the posterior mesentery, thus providing a secure grasp on the vas 50. The tip 518 pierces the very thick scrotal skin, and when the clamp is rolled onto its side, the very thick skin will be available to the surgeon to create the skin opening The surgeon may utilize the locking portions 120,122 to fix the position of the distal tips 516, 518 relative to each other. With the vas 50 secured, the surgeon may use a scalpel 60 on the vas 50. That is, the surgeon may position a scalpel blade in the gap between the proximal ends 324, 326 and engage the vas 50.

Accordingly, the method of using the surgical apparatus 10, an more specifically the preferred embodiment shown in FIG. 12, is preferred for an “open” vasectomy and its use includes the following steps: isolating 1000 the vas, making an incision 1002 in a scrotum adjacent the vas, positioning 1004 the clamping structure first component 17 on one side of the vas 50 within the scrotum, positioning 1006 said clamping structure second component 19 on the side of the vas 50 opposing the first component 17 within the scrotum, moving 1008 the surgical instrument into said closed position, wherein said clamping structure first component 17 pierces and engages one side of the vas 50 and said clamping structure second component 19 pierces and engages the side of the vas 50 opposing the first component 17, pulling 1010 the vas 50 partially from the scrotum, and cutting 1012 a portion of the vas 50 while the clamp secures the vas for the remainder of the occlusive process. As is known, cauterization is an equivalent of cutting the vas 50 and may be used for the occlusive process. As noted above, if the surgical apparatus 10 includes a clamping structure 15 with pointed tips 16, 18, then the step of moving 1008 the surgical instrument into said closed position, wherein said clamping structure first component 17 engages one side of the vas 50 and said clamping structure second component 19 engages the side of the vas 50 opposing the first component 17 includes the further step of: piercing 1009 the vas 50 with said clamping structure first and second component 17, 19.

As further noted above, it is desirable to not contact the vasal artery and the plurality of vessels within the posterior mesentary 52 during the clamping procedure. Thus, the said step of moving 1008 the surgical instrument into said closed position does not include the step of substantially encircling the entire vas 50. Put another way, said clamping structure first component 17 and said clamping structure second component 19 engage the vas 50 at a location spaced from the vasal artery 52. This configuration allows for the blood flow from the vasal artery 52 to the vas 50 to not be substantially disturbed during the step of moving 1008 the surgical instrument into said closed position.

Although certain embodiments of the present invention have been described with particularity, these embodiments are illustrative and do not limit the present invention. In particular, the present invention is not limited to a particular surgical procedure or size of device, nor to the type or vessel, duct, tube, organ or body structure that can be manipulated. Upon review of the foregoing, numerous adaptations, modifications, and alterations will occur to those skilled in the art. These will all be, however, within the spirit of the present invention. Accordingly, reference should be made to the appended claims in order to ascertain the true scope of the present invention.

What is claimed is:

1. Surgical apparatus comprising:
a first elongated member having a proximal end and a distal end;
a second elongated member having a proximal end and a distal end;
said first and second members rotatably coupled at a pivot point and movable between a closed position and an open position;
a clamping structure first component disposed at said first member distal end;
a clamping structure second component disposed at said second member distal end;
said clamping structure first component and said clamping structure second component structured to partially encircle a tubular body member; and
said clamping structure having a gap on the proximal side structured to allow a scalpel to pass therethrough when said first and second members are in said closed position.

2. The apparatus of claim 1 wherein said clamping structure gap is between about 0.5 mm and 3.0 mm.

3. The apparatus of claim 2 wherein said clamping structure gap is about 1.0 mm.

4. The apparatus of claim 1 wherein:
said clamping structure first component has a quarter-circular medial portion with an inwardly extending distal tip; and
said clamping structure second component has a quarter-circular medial portion with an inwardly extending distal tip.

5. The apparatus of claim 4 wherein:
said clamping structure first component distal tip is pointed;
said clamping structure second component distal tip is pointed;
said first component distal tip and said second component distal tip structured to pierce a vas deferens.

6. The apparatus of claim 5 wherein:
said clamping structure first component distal tip and said clamping structure second component distal tip each has a centerline; and
wherein said first component distal tip centerline and said second component distal tip are substantially aligned.

7. The apparatus of claim 5 wherein:
said clamping structure first component distal tip and said clamping structure second component distal tip each has a centerline; and
wherein said first component distal tip centerline and said second component distal tip are parallel and offset from each other.

8. The apparatus of claim 5 wherein:
said clamping structure first component distal tip and said clamping structure second component distal tip each has a centerline; and
wherein said first component distal tip centerline and said second component distal tip are angled relative to each other.

9. The apparatus of claim 1 wherein:
said clamping structure first component has a quarter-circular medial portion with an inwardly extending distal tip;
said clamping structure second component has a semicircular medial portion with arcuate distal tip;
said clamping structure first component distal tip is pointed;
said clamping structure second component distal tip is pointed; and
said first component distal tip and said second component distal tip structured to pierce a vas deferens.

10. The apparatus of claim 9 wherein:
said clamping structure first component distal tip and said clamping structure second component distal tip each has a centerline; and
wherein said first component distal tip centerline and said second component distal tip are parallel and offset from each other.

11. The apparatus of claim 1 wherein:
said clamping structure first component has a quarter-circular medial portion with an inwardly extending distal tip;
said clamping structure second component has a semicircular medial portion;
said clamping structure first component distal tip is pointed; and
said first component distal tip and said second component distal tip structured to pierce a vas deferens.

12. The apparatus of claim 1 wherein said clamping structure first component and said clamping structure second component structured to pierce opposing sides of a tubular body member;

13. The apparatus of claim 1 wherein said surgical apparatus is a vas clamp.

14. A method of performing a vasectomy using a surgical apparatus having a first elongated member having a proximal end and a distal end, a second elongated member having a proximal end and a distal end, said first and second members rotatably coupled at a pivot point and movable between a closed position and an open position, a clamping structure first component disposed at said first member distal end, a clamping structure second component disposed at said second member distal end, said clamping structure first component and said clamping structure second component structured to engage opposing sides of a tubular body member, said clamping structure having a gap on the proximal side structured to allow a scalpel to pass therethrough when said first and second members are in said closed position, said method comprising the steps:
isolating the vas;
making an incision in a scrotum above or over the vas;
positioning said clamping structure first component on one side of the vas within the scrotum;
positioning said clamping structure second component on the side of the vas opposing the first component within the scrotum;
moving the surgical instrument into said closed position, wherein said clamping structure first component engages one side of the vas and said clamping structure second component engages the side of the vas opposing the first component;
pulling the vas partially from the scrotum; and
cutting or cauterizing a portion of the vas as part of the occlusive process.

15. The method of claim 14 wherein, said clamping structure first component and said clamping structure second component are pointed and said step of moving the surgical instrument into said closed position, wherein said clamping structure first component engages one side of the vas and said clamping structure second component engages the side of the vas opposing the first component includes the further step of piercing the vas with said clamping structure first and second component.

16. The method of claim 15 wherein said clamping structure first component has an arcuate medial portion with an inwardly extending distal tip, said clamping structure second component has an arcuate medial portion with an inwardly extending distal tip and said step of moving the surgical instrument into said closed position does not include the step of substantially encircling the vas.

17. The method of claim 16 wherein said clamping structure first component and said clamping structure second component engage the vas at a location spaced from the vasal artery and the plurality of vessels within the posterior mesentery to the vas.

18. The method of claim 15 wherein said clamping structure first component has an arcuate medial portion with an inwardly extending distal tip, said clamping structure second component has an arcuate medial portion with an inwardly extending distal tip and wherein the blood flow from the vasal artery and the plurality of vessels within the posterior mesentary to the vas is not substantially disturbed during the step of moving the surgical instrument into said closed position.
